(19) United States
(12) Patent Application Publication
(10) Pub. No.: US 2026/0258172 A1
(43) Pub. Date: Sep. 3, 2026

(54) CATIONIC POLYMERS WITH SELECTIVE ANTIFUNGAL PROPERTIES

(71) Applicant: Uniwersytet Jagiellonski, Kraków (PL)

(72) Inventors: Magdalena SKÓRA, Kraków (PL); Kamil KAMINSKI, Kraków (PL)

(73) Assignee: Uniwersytet Jagiellonski, Kraków (PL)

(21) Appl. No.: 18/995,888

(22) PCT Filed: Jul. 17, 2023
(86) PCT No.: PCT/PL2023/050061

§ 371 Date: Jan. 17, 2025

(30) Foreign Application Priority Data
Jul. 17, 2022 (PL) P.441751

(51) Int. Cl. C08F 120/60; A01N 37/20; A01P 3/00

(52) U.S. Cl. CPC C08F 120/60; A01N 37/20; A01P 3/00

(57) ABSTRACT

Cationic polymers are disclosed, prepared based on the [2-(methacryloxy)ethyl]trimethylammonium chloride monomer, characterized by selective antifungal activity and low toxicity against mammalian cells, in particular human cells which, as a result, are particularly useful for manufacturing antifungal drugs and formulations.

The invention concerns cationic polymers prepared based on the [2-(methacryloxy)ethyl]trimethylammonium chloride (hereinafter also referred as PMAPTAC) monomer, characterized in selective antifungal activity and low toxicity toward mammalian, in particular human, cells. Therefore, they may be potentially used in formulations having antifungal effects, such as in antifungal drugs, cosmetics and plant protection products.

Cationic monomers, such as [2-(methacryloxy)ethyl]trimethylammonium chloride (hereinafter also referred as PMAPTAC) are disclosed in patent application P.402516, prepared through the quaternization of 2-dimethylaminoethylmethacrylate using methyl chloride. In addition, it is disclosed that the cationic monomer has fungistatic properties against all Candida albicans, Candida krusei and Candida parapsilosis strains. However, the usefulness of the polymers is not disclosed and the possibility of preparing specific polymers having selective antifungal properties is not suggested.

The antibacterial activity of MAPTAC is disclosed in Colloids and Surfaces A 549 (2018) 122-129.

The antibacterial activity of MAPTAC and copolymers thereof is disclosed in Progress in Polymer Science 39 (2014) 1096-1143.

The antibacterial activity of MAPTAC in a form incorporated in acrylic resins is disclosed in Int J Artif Organs 2012; 35 (10): 854-863.

The objective of the invention is to provide a substance characterized in selective antifungal activity and low toxicity toward mammalian, in particular human, cells.

The objective has surprisingly been achieved owing to the present invention.

The object of the invention is a cationic polymer with mean molecular weight above 13 kDa prepared based on [2-(methacryloxy)ethyl]trimethylammonium chloride monomer having a formula:

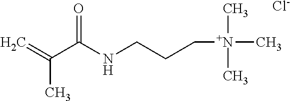

useful in the treatment or prevention of diseases caused by fungi.

The cationic polymer for use of the invention preferably has mean molecular weight between 13 kDa and 892 kDa, preferably between 29 kDa and 892 kDa.

The fungus is preferably of a species selected from: dermatophytes of the genus Trichophyton, in particular Trichophyton interdigitale, Trichophyton mentagrophytes, Trichophyton tonsurans, Trichophyton rubrum, Scopulariopsis brevicaulis, species of the genus Fusarium, in particular Fusarium graeminarum, Fusarium oxysporum, Fusarium proliferatum, Fusarium solani, Fusarium verticilloides, Scedosporium apiospermum, Lomentospora prolificans, Candida parapsilosis, Cryptococcus neoformans.

To ensure better understanding of the core of the invention, this specification is illustrated by the attached figures.

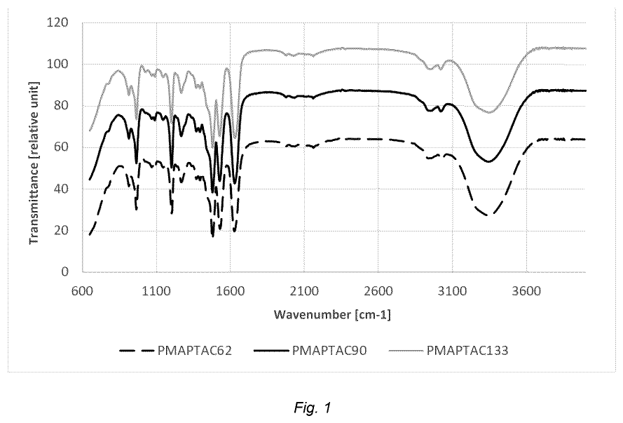

FIG. 1 shows FT-IR spectra of the disclosed polymers.

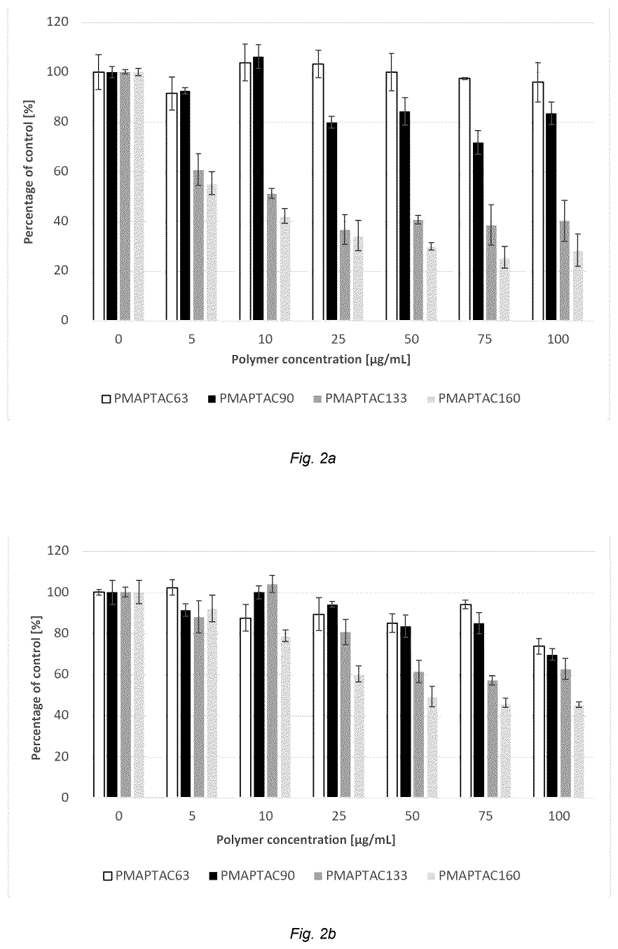

FIG. 2 shows the effect of the polymers tested on the survival of 3T3-L1 fibroblast cells in experiments performed using a serum-free medium (FIG. 2a) and a medium with serum added (FIG. 2b).

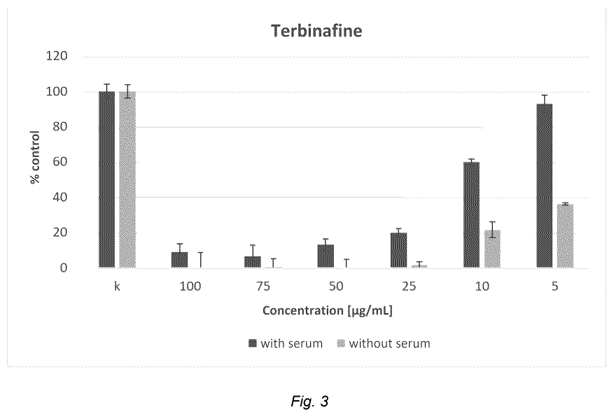

FIG. 3 shows the assessment of terbinafine toxicity.

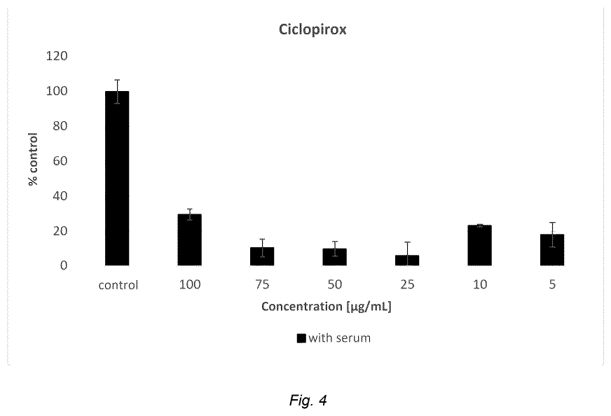

FIG. 4 shows the assessment of ciclopirox toxicity.

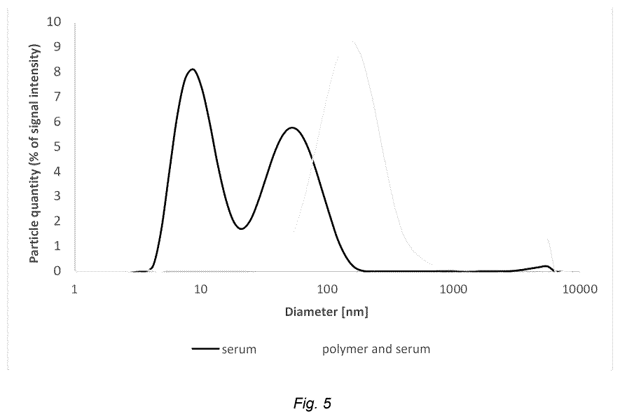

FIG. 5 shows particle size distribution in mixtures of polymer solutions with serum and serum alone.

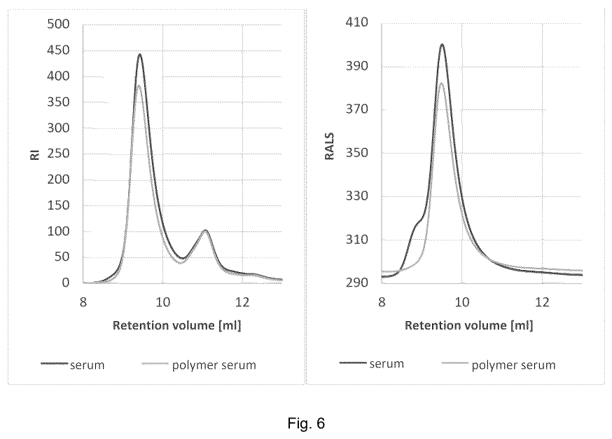

FIG. 6 shows chromatograms of centrifuged mixtures of serum and polymer solutions. A refraction detector and a light scattering detector are shown on the left and on the right, respectively.

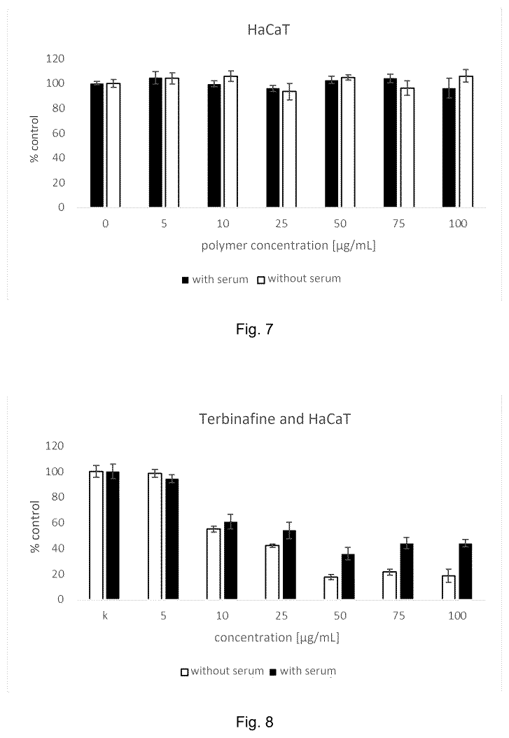

FIG. 7 shows the effect of the polymer on the viability of keratinocytes from the HaCaT cell line in experiments performed using a medium with serum and a serum-free medium.

FIG. 8 shows the effect of terbinafine on the viability of keratinocytes from the HaCaT cell line in experiments performed using a medium with serum and a serum-free medium.

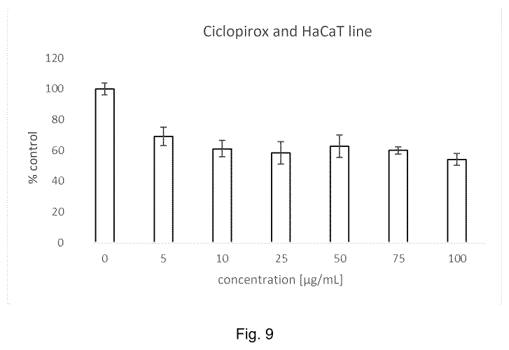

FIG. 9 shows the effect of ciclopirox on the viability of keratinocytes from the HaCaT cell line in experiments performed using a medium with serum.

In addition, the core of the invention is clarified in the examples below.

Examples 1, 2 and 4 disclose successive stages of an example embodiment of the process of the invention, and examples 3 and 5 disclose the properties of the materials obtained according to the invention.

### Synthesis of Polymers

To effectively compare the effect of molecular weight of cationic macromolecules on biological activity, four syntheses were completed with differing reaction times or initiator quantities in two variants. The first one used an inverter (RAFT controlled polymerization), and the other one was classic (uncontrolled) free-radical polymerization. RAFT controlled polymerization in the presence of the inverter was used to synthesize macromolecules with lower mass than obtainable in classic uncontrolled systems. The synthesis was completed as described below.

### Example 1. Polymerization of Lower-Weight Polycations

First, the polymerization inhibitor was removed from MAPTAC monomer solution (50% in water) by passing the mixture through neutralized aluminum oxide. Subsequently, 6 mL of the MAPTAC monomer solution, 15 mg V-501 polymerization initiator (4,4′-azobis(4-cyanovaleric) acid) and 5 mL water was mixed. The mixture was heated to 40° C. and degassed by passing argon therethrough for about 0.5 h. Subsequently 34.66 mg CPD (4-cyano-4-(phenylcarbonothioylthio) pentanoic) acid dissolved in 1 mL methanol was added to the solution. The reaction vessel was placed on a magnetic stirrer and stirred. Polymerization was conducted for 7 h (PMAPTAC62) and 24 h (PMAPTAC90) at 70° C. The solution containing the synthesis product was cooled. The next step was dialysis against distilled water in a dialysis tube (mass cutoff value: below 3.5 kDa) for 5 days while replacing water every 24 h. The polymer after dialysis was isolated from the solution by freeze-drying.

### Example 2. Polymerization of Higher-Weight Polymers

First, the polymerization inhibitor was removed from MAPTAC monomer solution (50% in water) by passing the mixture through neutralized aluminum oxide. Subsequently, 6 mL of the MAPTAC monomer solution and 15 mg (PMAPTAC133) or 30 mg (PMAPTAC160) V-501 polymerization initiator and 5 mL water was mixed. The mixture was heated to 40° C. and degassed by passing argon therethrough for about 0.5 h. The reaction vessel was placed on a magnetic stirrer and stirred. Polymerization was conducted in either case for 4 h at 70° C. The solution containing the synthesis product was cooled. The next step was dialysis against distilled water in dialysis a tube for 5 days while replacing water every 24 h. The polymer after dialysis was isolated from the solution by freeze-drying.

### Physicochemical Parameters of the Polymers Tested

### Example 3. General Physical Characteristics of the Polymers

The data collected here were to confirm that polycations were obtained and their molecular weight was determined. As the list below shows, four polycations were obtained (as evidenced by the positive zeta potential value), differing in their mean molar mass (determined based on GPC measurements).

TABLE 1
General characteristics of the polymers tested

| Name of | Mean molecular | Polymerization | Zeta potential in |
|---|---|---|---|
| polymer | weight, Mp [kDa] | method | water* [mV] |
| PMAPTAC62 | 13.76 | RAFT | 31.83 ± 5.35 |
| PMAPTAC90 | 19.93 | RAFT | 41.20 ± 5.17 |
| PMAPTAC133 | 29.54 | Radical | 23.03 ± 4.25 |
| PMAPTAC160 | 892 | Radical | 65.7 ± 3.72 |

*concentration of polymer solution: 2 g/L

### Example 4. Chemical Characteristics

The data below confirm that the expected polymer chemical structures were obtained in the syntheses. This is confirmed by the similar shape of FT-IR spectra (see FIG. 1) and the elemental composition.

TABLE 2
Elemental composition of the resulting polymers.

| Name of | | | | | |
|---|---|---|---|---|---|
| polymer | N | C | H | S | N/C |
| PMAPTAC62 | 10.24 | 44.30 | 9.72 | 0 | 0.2311 |
| PMAPTAC90 | 10.58 | 45.61 | 9.99 | 0 | 0.2319 |
| PMAPTAC133 | 10.45 | 47.07 | 9.94 | 0 | 0.2219 |
| PMAPTAC160 | 10.46 | 47.51 | 9.95 | 0 | 0.2201 |

IR spectra show the presence of all functional groups as predicted, to be found in the polymerization product of the MAPTAC monomer. The peak attributed to vibrations of the methyl groups in the quaternary amine responsible for the positive charge of the macromolecule at 1485 cm-1 is particularly important at this point.

### Example 5. Preliminary Assessment of Toxicity in Cell Lines

### Tissue Fibroblast Line

Preliminary tests were conducted in the 3T3-L1 murine embryonic fibroblast line (ATCC, CL-173™), an immortalized line (highly tolerant cells). DMEM was used as the medium. 24 h after seeding and successful adhesion, polymers dissolved in the medium were added to the cells (the medium was first replaced with a serum-free medium in serum-free experiments), and viable cells were quantified after another 24 h using the neutral red uptake assay [1]. The results are shown in FIG. 2.

The tests showed that the larger polymer mass the higher the negative effect on the cells. The number of viable cells decreased to less than 50% only for the highest mass polymer (PMAPTAC160) with a concentration of 50 μg/mL and higher in the presence of serum. Higher toxicity was found in serum-free systems. Viable cell counts reduced to more than 50% occurred for two polymers: PMAPTAC133 and PMAPTAC160, and concerned concentrations equal to or greater than 25 μg/mL and 10 μg/mL, respectively. For the PMAPTAC133 polymer and a concentration range of 25-100 μg/mL, the viable cell count did not decrease below 40%, which showed that the polymer was moderately toxic toward the test cells. A larger-mass polymer, i.e. PMAPTAC160, caused the viable cell count to be reduced to 25% of the control value in the same concentration range.

It is concluded based on the results that PMAPTAC160, the polymer with the highest molecular weight, was too toxic for further investigation even if extracorporeal application was envisaged, while lower-mass polymers had moderate and acceptable toxicity, especially with extracorporeal application.

The toxicity of ciclopirox and terbinafine, two commercially used antifungal drugs, was evaluated in similar conditions (the same cell line, medium composition and exposure time).

The obtained results of toxicity testing for terbinafine and ciclopirox are shown in FIG. 3 and FIG. 4, respectively. It is found when comparing the results from those obtained for the polymers that all the macromolecules tested were significantly less toxic than low-molecular weight drugs. For terbinafine and experiments in the medium with serum, considered the most reliable here, a concentration of 25 μg/mL caused the cell count to decrease to below 50%. The decrease was significant, because the cell count did not exceed 10% for concentrations of 50, 75 and 100 μg/mL, and values above 40% were obtained for the polymers. Ciclopirox caused death of more than half of the cells at concentrations as low as 5 μg/mL. Based on the strong interaction of the drug with serum, the experiment in the serum-free medium would have been unreliable and it was not performed [2].

Preliminary Evaluation of Toxicity of the Polymers after Intravenous Application: Interaction with Serum Proteins

Because the most promising results in terms of antimycotic properties were obtained for the PMAPTAC133 polymer, interactions with serum proteins were tested for the polycation.

### Example 6. Generation of Submicron-Sized Particles in the Reaction Between the PMAPTAC133 Polycation and Serum Proteins which May Obstruct Blood Vessels

The most serious risk related to polymers interacting with serum proteins (which is frequently the case for polycations) is the generation of micron- and submicron-sized particles in the reactions as they could obstruct the lumen of capillary blood vessels. The size of the products was assessed for the polymer tested using dynamic light scattering. In the experiment, equal volumes of 5 g/L polymer solution in 0.1 M NaCl and bovine serum (the sera and the polymer solution were centrifuged at 10,000 rpm for 5 minutes immediately before mixing) were mixed. A similar mixture prepared from serum and 1 M NaCl solution without the polymer served as the control. The results are shown in FIG. 5.

The conclusion based on the results is that interaction with serum proteins occurs for the polymer and, as a result, submicron particles form, but their size does not exceed 1 μm; therefore, the risk of capillary embolism following intravenous administration is considered low. An optimum situation would be if the interaction had not been observed at all, but the present case does not rule out the application of the novel compound as a drug because commercially used antifungal drugs, that is, ciclopirox and terbinafine, also show strong interaction with serum proteins as reported in the literature. However, this is an argument for using the polymer mainly as an extracorporeal product.

### Example 7. Changes in Serum Protein Composition Due to Interactions with the Polycation

As discussed above, processes associated with serum protein binding by polymers may affect the composition of the protein mixture (after separation of reaction products). Therefore, it was attempted to evaluate the specific serum components that react with the test polymer (PMAPTAC133) based on changes in serum composition after separation of reaction products by centrifugation at 10,000 rpm for 5 min. GPC was used to evaluate changes in protein mixture composition as it measures molecular weights of macromolecules found in a mixture while determining their quantities. The eluent was 0.1 M NaCl solution, and the serum before the measurement was mixed with the eluent in proportions of 0.05 mL and 0.95 eluent or eluent with the polymer with a concentration of 5 g/L. The injection volume was 100 μL, and the flow rate was 0.8 mL/min. Results are shown in FIG. 6.

The polymer interacts with serum proteins and results in decreased concentrations of proteins having the highest content in the mixture and having the highest molecular weights, that is, albumins. This is shown by the decreased signals for both detectors at the shortest retention times, which corresponds to the largest molecular weights. Observations from the last two points show that intravenous use of PMAPTAC133 could be dangerous. This is not a practical problem, though, because the compounds are meant for extracorporeal use for treatment of skin, nail and hair mycosis.

### Example 8. Testing the Antifungal Activity of the Polymers

In vitro tests of the antifungal activity of the polymers were performed with respect to selected fungal species obtained from international culture collections and the microorganism collection in the Department of Infection Control and Mycology, Chair of Microbiology, Jagiellonian University Medical College. The test was performed using the microdilution method in a liquid medium on flat-bottomed, 96-well polypropylene titration plates. The applied methodology was based on antifungal susceptibility testing procedures developed by the European Committee on Antimicrobial Susceptibility Testing (EUCAST) [3, 4] and on the Hancock Lab procedure for antimicrobial cationic peptides [5].

The test compounds were dissolved in sterile distilled water and starting concentrations of 5 g/L were prepared, from which a series of two-fold dilutions in water were obtained. Respective polymer dilutions were transferred in a volume of 20 μL on titration plates (wells 1-10). 20 μL of sterile distilled water was transferred to each of wells 11 and 12. Wells 1-11 were inoculated with fungi in a volume of 180 μL of fungal cell suspensions in a liquid medium, and 180 μL of the sterile medium was transferred to well 12. Fungal suspensions were prepared from fresh cultures on agar media by collecting spores using a sterile swab into sterile distilled water or sterile distilled water with Tween-20 added. Suspensions of certain strains were filtered to remove any hyphal elements (filters with a pore diameter of 20 μm and 10 μm were used). The suspensions were brought to a density of 0.5 on the McFarland scale, and subsequently diluted to 1:20 in a liquid RPMI-1640 medium with L-glutamine and without sodium bicarbonate, supplemented with glucose (2%) and buffered morpholinopropanesulfonic acid (MOPS) (0.165 mol/L).

Final polymer concentrations on plates after adding fungal suspensions in the liquid medium were 0.49-250 mg/L or 0.98-500 mg/L.

Incubation was performed at room temperature, 27° C., 37° C., depending on the fungal species, until fungal growth was obtained in growth control (well 11). After the incubation period, fungal growth was evaluated visually and the lowest polymer concentrations that caused complete fungal growth inhibition (MIC (minimal inhibitory concentration) values) were determined. The results obtained in several independent tests are listed in Tables 3-5.

Preliminary tests showed that antimycotic activity was related with the molecular weight of a polymer. It was shown that polymers with the highest molecular weights (PMAPTAC133, PMAPTAC160) had better in vitro antifungal activity (lower MIC values) than other polymers (Table 3). Considering the antifungal properties of PMAPTAC133 shown and its relatively low cell toxicity (see the results shown above), the compound was subjected to more extensive tests using a greater number of fungal strains.

The list of species tested with the number of strains tested and resulting MIC values for the PMAPTAC133 polymer is shown in Table 4. The lowest MIC values were obtained for dermatophytes of the genus Trichophyton (Trichophyton interdigitale, Trichophyton mentagrophytes, Trichophyton tonsurans, Trichophyton rubrum), molds Scopulariopsis brevicaulis, Lomentospora prolificans, Scedosporium apiospermum and Fusarium graeminarum, Fusarium oxysporum, Fusarium proliferatum, Fusarium solani, Fusarium verticilloides and yeasts of the species Candida parapsilosis and Cryptococcus neoformans.

The effect of the PMAPTAC133 polymer on Scopulariopsis brevicaulis was confirmed for several dozen strains, including both those from international culture collections and clinical strains isolated from nails and skin and deposited in the Department of Infection Control and Mycology, Jagiellonian University Medical College (Tables 4 and 5). As a result, it can be concluded with high certainty that the new polymer has antifungal properties against the species Scopulariopsis brevicaulis.

It was also shown that the PMAPTAC133 polymer had an inhibitory effect on the growth of members of Mucorales (species of the genus Mucor, Actinomucor, Rhizopus, Cunninghamella were tested) (Table 4), but the activity was weaker and the polymer only caused a decrease in fungal growth in the concentration range tested (between 0.49 mg/L and 500 mg/L) compared to control growth. A similar effect was observed for the species Candida tropicalis.

No antifungal effect of the PMAPTAC133 polymer was observed in the species Candida albicans, Candida auris, Candida krusei, Candida glabrata, Botrytis cinerea, Beauveria bassiana, while the activity against strains of the genus Aspergillus seems to be variable, at least depending on the species, and we have not shown an antifungal effect in our tests performed so far on the most common human pathogens of the genus, that is, Aspergillus flavus and Aspergillus fumigatus (Tables 4 and 5).

TABLE 3
Lowest concentration values of PMAPTAC62, PMAPTAC90,
PMAPTAC133, PMAPTAC160 which inhibit the growth
of selected fungal species (MIC).

| | MIC values for the polymers [mg/L] | | | |
|---|---|---|---|---|
| Species | PMAPTAC62 | PMAPTAC90 | PMAPTAC133 | PMAPTAC160 |
| Candida albicans | >500 | >500 | >500 | >250 |
| ATCC 90028 | | | | |
| Candida krusei | >500 | >500 | >500 | >250 |
| ATCC 6259 | | | | |
| Aspergillus flavus | >500 | >500 | >500 | >250 |
| ATCC 204304 | | | | |
| Aspergillus | >500 | >500 | >500 (3.91 mg/L, | >250 (3.91 mg/L, |
| brasiliensis | | | clear growth | clear growth |
| ATCC 16404 | | | suppression) | suppression |
| Penicillium | >500 | >500 | >500 (7.81 mg/L, | |
| funiculosum | | | clear growth | |
| DSM 10640 | | | suppression) | |
| Trichophyton | >500 | >500 | 1.95 (0.98 mg/L, | 0.98 |
| mentagrophytes | | | clear growth | |
| ATCC 18484 | | | suppression) | |
| Fusarium solani | >500 | >500 | 1.95 | 1.95 |
| strain 155 | | | | |
| Scopulariopsis | 0.98 | 0.98 | 0.98 | —* |
| brevicaulis | | | | |
| strain 26 | | | | |

*Results were obtained for other strains of the species, in a range of 0.98-1.95 mg/L.

TABLE 4
Minimum concentration values of the PMAPTAC133 polymer which inhibit the growth
of selected fungal species (MIC) obtained in several independent experiments.

| | Number | | | | |
|---|---|---|---|---|---|
| | of strains | MIC range | MIC50 | MIC90 | Mode |
| Species | tested | [mg/L] | [mg/L] | [mg/L] | [mg/L] |
| Actinomucor elegans | 1 | >250 | — | — | — |
| | | (15.62-31.25 | | | |
| | | mg/L, growth | | | |
| | | suppression) | | | |
| Aspergillus brasiliensis | 1 | >500 | — | — | — |
| Aspergillus calidoustus | 1 | 3.91 | — | — | — |
| Aspergillus flavus | 4 | >500 | >500 | >500 | >500 |
| Aspergillus fumigatus | 7 | 250->500 | >500 | >500 | >500 |
| Aspergillus nidulans | 1 | 3.91 | — | — | — |
| Aspergillus niger | 5 | >500 | >500 | >500 | >500 |
| Aspergillus lentulus | 1 | >250 | — | — | — |
| Aspergillus ochraceus | 2 | >500 | — | — | — |
| Aspergillus terreus | 1 | >500 | — | — | — |
| Aspergillus ustus | 2 | 1.95-3.91 | — | — | — |
| Aspergillus verrucosum | 2 | 1.95->500 | — | — | — |
| Aspergillus versicolor | 2 | 1.95->500 | — | — | — |
| Aspergillus sydowii | 2 | 3.91->500 | — | — | — |
| Candida albicans | 1 | >500 | — | — | — |
| Botrytis cinerea | 1 | >250 | — | — | — |
| Beuveria bassiana | 1 | >250 | — | — | — |
| Candida auris | 1 | >250 | — | — | — |
| Candida glabrata | 1 | >250 | — | — | — |
| Candida krusei | 1 | >500 | — | — | — |
| Candida parapsilosis | 2 | 3.91-7.81 | — | — | — |
| Candida tropicalis | 1 | >250 | — | — | — |
| | | (15.62 mg/L, | | | |
| | | clear growth | | | |
| | | suppression) | | | |
| Cryptococcus neoformans | 3 | 1.95-7.81 | 3.91 | 3.91 | 3.91 |
| Cunninghamella | 1 | >250 | — | — | — |
| blakesleeana | | (62.5 mg/L, | | | |
| | | growth | | | |
| | | suppression) | | | |
| Fusarium graeminarum | 1 | 3.91 | — | — | — |
| Fusarium oxysporum | 1 | 1.95-7.81 | — | — | — |
| Fusarium proliferatum | 1 | 1.95 | — | — | — |
| Fusarium solani | 1 | 0.98-3.91 | — | — | — |
| Fusarium verticilloides | 1 | 1.95-3.91 | — | — | — |
| Lomentospora prolificans | 2 | 0.49-0.98 | — | — | — |
| Microsporum canis | 1 | 1.95 | — | — | — |
| Mucor indicus | 1 | >250 | — | — | — |
| | | (15.62-31.25 | | | |
| | | mg/L, growth | | | |
| | | suppression) | | | |
| Mucor mucedo | 1 | 31.25->250 | — | — | — |
| | | (31.25, growth | | | |
| | | suppression) | | | |
| Mucor flavus | 1 | >250 | — | — | — |
| | | (125 mg/L, | | | |
| | | growth | | | |
| | | suppression) | | | |
| Mucor variisporus | 1 | 15.62 | — | — | — |
| Mucor amphibiorum | 1 | >250 | — | — | — |
| | | (31.25 mg/L, | | | |
| | | growth | | | |
| | | suppression) | | | |
| Mucor racemnosus | 1 | >250 | — | — | — |
| | | (31.25 mg/L, | | | |
| | | growth | | | |
| | | suppression) | | | |
| Mucor hiemalis | 1 | >250 | — | — | — |
| | | (62.5 mg/L, | | | |
| | | growth | | | |
| | | suppression) | | | |
| Penicillium funiculosum | 1 | >500 | — | — | — |
| Rhizopus oryzae | 1 | >250 | — | — | — |
| | | (15.62-62.5 mg/L, | | | |
| | | growth | | | |
| | | suppression) | | | |
| Scedosporium | 2 | 0.98 | — | — | — |
| apiospermum | | | | | |
| Scopulariopsis brevicaulis | 49 | 3.91-7.81 | 3.91 | 7.81 | 3.91 |
| Trichophyon | 2 | 1.95-3.91 | — | — | — |
| mentagrophytes | | | | | |
| Trichophyton interdigitale | 7 | 1.95-3.91 | 3.91 | 3.91 | 3.91 |
| Trichophyton rubrum | 1 | 500 | — | — | — |
| Trichophyton tonsurans | 3 | 1.95-3.91 | 1.95 | 3.91 | 1.95 |

MIC—minimal inhibitory concentration related to fungal growth,
MIC50—minimal inhibitory concentration related to the growth of 50% of the fungal strains tested,
MIC90—minimal inhibitory concentration related to the growth of 90% of the fungal strains tested

### Example 9. Comparison of Antifungal Activity of the PMAPTAC133 Polymer and Antifungal Drugs (Terbinafine, Ciclopirox)

A test was performed on selected fungal strains to compare MIC values of the PMAPTAC133 polymer with the MIC values of two known antifungal drugs (terbinafine and ciclopirox), used in the treatment of fungal infections of glabrous skin, hairy skin and nails.

The procedure described in the section above (Example 8) was used in the testing.

The drugs were dissolved in dimethyl sulfoxide (DMSO) and starting concentrations of 4 g/L were prepared, from which series of two-fold dilutions in DMSO were obtained. Respective polymer concentrations were diluted to 1:100 in sterile water and subsequently transferred to wells 1-10 on a titration plate in a volume of 20 μL. Wells 11 and 12 were filled up with 20 μL DMSO diluted to 1:10 in water. Wells 1-11 were inoculated with fungi in a volume of 180 μL of fungal cell suspensions in a liquid medium, and 180 L of the sterile medium was transferred to well 12. Final drug concentrations on plates after adding fungal suspensions in the liquid medium were 0.008-4 mg/L.

It was found that MIC values for the polymer were lower than for terbinafine and ciclopirox against the test strains of the species Scopulariopsis brevicaulis, Fusarium solani, Fusarium oxysporum, and they were lower for the polymer than for ciclopirox for dermatophytes Trichophyton mentagrophytes, Trichophyton interdigitale, Trichophyton tonsurans (Table 5).

TABLE 5
Minimum inhibitory concentration (MIC) values for fungal growth [mg/L] obtained
for the PMAPTAC133 polymer, ciclopirox and terbinafine in a comparative test.

| | incubation time | | | | | | | | |
|---|---|---|---|---|---|---|---|---|---|
| Fungal | PMAPTAC133 | | | Ciclopirox | | | Terbinafine | | |
| species | 24 h | 48 h | 72 h | 24 h | 48 h | 72 h | 24 h | 48 h | 72 h |
| Aspergillus | >250 (3.91, | >250 (3.91, | — | >4 | >4 | — | 2 | >4 (2, | — |
| brasiliensis | clear growth | clear growth | | | | | | growth | |
| ATCC 16404 | suppression) | suppression) | | | | | | suppression) | |
| Aspergillus | >250 | >250 | — | >4 | >4 | — | 4 | >4 (2, | — |
| flavus | | | | | | | | growth | |
| ATCC 204304 | | | | | | | | suppression) | |
| Aspergillus | >250 | >250 | — | >4 | >4 | — | >4 (2, | >4 | — |
| fumigatus | | | | | | | growth | | |
| DSM 819 | | | | | | | suppression) | | |
| Aspergillus | >250 | >250 | — | >4 | >4 | — | 4 | >4 (4, | — |
| fumigatus | | | | | | | | growth | |
| SU 56 | | | | | | | | suppression) | |
| Aspergillus | >250 | >250 | — | >4 | >4 | — | 2 | >4 (2, | — |
| terreus | | | | | | | | growth | |
| DSM 1958 | | | | | | | | suppression) | |
| Candida | >250 | >250 | — | >4 | >4 | — | >4 | >4 | — |
| albicans | | | | | | | | | |
| ATCC 90028 | | | | | | | | | |
| Candida | >250 | >250 | — | >4 | >4 | — | >4 | >4 | — |
| glabrata | | | | | | | | | |
| ATCC 15545 | | | | | | | | | |
| Candida krusei | >250 | >250 | — | >4 | >4 | — | >4 | >4 | — |
| ATCC 6258 | | | | | | | | | |
| Cryptococcus | 1.95 | 3.91 (1.95, | | >4 (4, slight | >4 | — | >4 | >4 | — |
| neoformans | | marginal | | growth | | | | | |
| | | growth) | | suppression) | | | | | |
| Fusarium | 1.95 | 3.91 (1.95, | — | >4 | >4 | — | >4 | >4 | — |
| oxysporum | | marginal | | | | | | | |
| 109 | | growth) | | | | | | | |
| Fusarium | 1.95 | 3.91 (1.95, | — | >4 | >4 | — | >4 | >4 | — |
| oxysporum | | marginal | | | | | | | |
| DSM 841 | | growth) | | | | | | | |
| Fusarium | 1.95 | 3.91 | — | >4 | >4 | — | >4 | >4 | — |
| solani 155 | | | | | | | | | |
| Fusarium | 1.95 | 1.95 | — | >4 | >4 | — | >4 | >4 | — |
| solani | | | | | | | | | |
| DSM 1164 | | | | | | | | | |
| Scopulariopsis | 1.95 | 3.91 (1.95, | — | >4 | >4 | — | >4 | >4 | — |
| brevicaulis 34 | | marginal | | | | | | | |
| | | growth) | | | | | | | |
| Scopulariopsis | 1.95 | 1.95 | — | >4 | >4 | — | >4 | >4 | — |
| brevicaulis | | | | | | | | | |
| DSM 9122 | | | | | | | | | |
| Trichophyton | 1.95 | 1.95 | 1.95 | >4 | >4 | >4 | slight | 0.5/0.25 | 1 |
| interdigitale | | | | | | | growth | | |
| 251 | | | | | | | | | |
| Trichophyton | 1.95 | 1.95 | 1.95 | >4 | >4 | >4 | slight | 0.25 | 0.5 |
| interdigitale | | | | | | | growth | | |
| D182 | | | | | | | | | |
| Trichophyton | ng | 1.95 | 1.95 | >4 | >4 | >4 | ng | 0.25 | 0.25 |
| interdigitale | | | | | | | | | |
| DSM4167 | | | | | | | | | |
| Trichophyton | 1.95 | 1.95 | 3.91 (1.95, | >4 | >4 | >4 | 0.125 | 0.5 (0.25, | 1 (0.25 |
| mentagrophyte | | | marginal | | | | | marginal | and 0.5, |
| ATCC 18484 | | | growth) | | | | | growth) | marginal |
| | | | | | | | | | growth) |
| Trichophyton | ng | >250 (slight | >250 (1.95, | ng | >4 | >4 | ng | 0.125 | 0.25 |
| rubrum | | growth) | clear growth | | | | | | |
| D197 | | | suppression) | | | | | | |
| Trichophyton | >250 (very | >250 | >250 | >4 | >4 | >4 | ng | 0.125 | 2 |
| rubrum | slight | | | | | | | | |
| D320 | growth) | | | | | | | | |
| Trichophyton | 3.91 (very | 7.81/3.91 | 7.81 | >4 | >4 | >4 | 0.06 (very | 0.25 | 0.25 |
| rubrum | slight | | | | | | slight | | |
| DSM 16111 | growth) | | | | | | growth) | | |
| Trichophyton | ng | 1.95 | 1.95 | ng | >4 | >4 | ng | 0.5 | 0.5 |
| tonsurans | | | | | | | | | |
| 264 | | | | | | | | | |
| Trichophyton | ng | 1.95 | 1.95 | ng | >4 | >4 | ng | 0.25 | 0.25 |
| tonsurans | | | | | | | | | |
| DSM 12285 | | | | | | | | | |

ng: no growth of cultured strain in positive control well

### Example 10. Evaluation of the Toxicity of a Polymer with a Mean Molecular Weight of 892 kDa in Keratinocytes

The testing was performed in the HaCaT cell line, a nontumorigenic, immortalized human keratinocyte line. The DMEM medium was used in the culture, including a modification with 10% fetal bovine serum added. 24 h after seeding and successful adhesion, the polymer dissolved in the medium was added to the cells (the medium was first replaced with a serum-free medium in serum-free experiments), and viable cells were quantified after another 24 h using the neutral red uptake assay [1]. The results are shown in FIG. 7.

The testing showed that the polymer with a mean molecular weight of 892 kDa was not toxic against keratinocytes. No reduction in viable keratinocyte cells incubated in the presence of the polymer at concentrations of up to 100 μg/mL was observed, irrespective of whether serum was present or not.

The toxicity of terbinafine and ciclopirox, two commercially used antifungal drugs, was evaluated in similar conditions (the same cell line, medium composition and exposure time). As for ciclopirox, the serum-free medium was not used because the drug strongly interacts with serum and testing in a serum-free medium would have been unreliable [2]. The results are shown in graphs in FIG. 8 and FIG. 9.

It was found that both terbinafine and ciclopirox were more toxic against keratinocytes than the polymer with a mean molecular weight of 892 kDa. At concentrations equal to or greater than 10 μg/mL, terbinafine caused reduction of viable keratinocytes of at least 40% compared to the control. As for a serum-free culture, concentrations of ≥50 μg/mL caused death of more than 70% of keratinocytes. The reduction of viable cells was lower in a culture in a medium with serum added. Already at a concentration of 5 μg/mL, ciclopirox caused reduction of viable keratinocyte cells by 30% compared to the control. Greater concentrations (>10 μg/mL) resulted in the reduction of keratinocytes of approx. 60%.

### CONCLUSIONS

It was found based on the testing completed that the cationic polymers prepared based on [2-(methacryloxy)ethyl]trimethylammonium chloride monomer had antifungal activity. It was unexpectedly found that the activity increased with an increasing mean molecular weight of the polymers. The results discussed above confirm activity of the polymers with a mean molecular weight greater than 13 kDa, and enhanced and broader antifungal activity was reported for polymers with a mean molecular weight greater than 29 kDa (Example 8, Table 3).

Preliminary tests showed that the cytotoxicity of the polymers against 3T3-L1 murine embryonic fibroblasts increased with increasing mean molecular weight (Example 5). However, subsequent tests in HaCaT keratinocyte cells showed that the toxicity of polymers with a weight of up to 892 kDa was negligible (Example 10, FIG. 7) and it was concluded that the polymers tested had variable cytotoxicity against different cell types. This could be used when selecting a polymer optimal for a specific application.

Considering the preliminary cytotoxicity testing results for mammalian cells, the PMAPTAC133 polymer with a mean molecular weight of 29.54 kDa was selected for further tests of antifungal activity. The tests confirmed its broad antifungal activity against various fungal species (Table 4).

The antifungal spectrum of activity includes species that cause pathologies in humans, animals and plants and, therefore, the present invention may be useful for treating or preventing diseases caused by the fungi.

Fungi of the species Trichophyon mentagrophytes, Trichophyton interdigitale, Trichophyton rubrum, Trichophyton tonsurans cause skin and nail infections in humans [6,7]. The species Trichophyton mentagrophytes also causes skin infections in animals [8, 9].

The species Scopulariopsis brevicaulis, one of the most common causes of non-dermatophyte mold onychomycosis [10], also causes infections of the skin [11], subcutaneous tissue [12], cornea and deep infections [14, 15] in humans. Furthermore, infections in animals have been reported [16, 17].

Fungi of the genus Fusarium, in particular the species Fusarium oxysporum and Fusarium solani, cause deep infections [18, 19], infections of the cornea and intraocular infections as well as infections of keratin-rich tissues [22, 23] in humans. They also cause infections in animals [24, 25].

The species Scedosporium apiospermum and Lomentospora prolificans cause skin and deep tissue infections in humans [26, 27]. Scedosporium apiospermum also causes infections in animals [28].

The species Candida parapsilosis is a cause of candidiasis affecting the skin, mucosae and deep infections, including blood infections in humans and animals [29-31].

The species Cryptococcus neoformans causes deep infections, including central nervous system infections in humans and animals, as well as superficial infections [32-34].

Fungal species of the genus Fusarium, in particular Fusarium graminearum and Fusarium verticillioides, are also dangerous pathogens that attack plants. The fungi cause for example fusarium ear blight, one of the most common and devastating diseases of wheat which causes serious losses due to reduced grain quality and quantity [35, 36], or corn infections [37]. Therefore, the invention can be useful for treating and preventing diseases caused by fungi in plants, in particular crop or ornamental plants.

Fusarium strains are also able to produce toxins dangerous to the health and life of humans and animals; when plants are infected by the fungi, they may contaminate plant-based foodstuffs and contribute to diseases in humans and animals known as mycotoxicoses [38-40]. This is another argument for using the invention in an agent for protecting plant, animal or human organisms against fungi.

### REFERENCES

[1] Repetto, G., del Peso, A. & Zurita, J. Neutral red uptake assay for the estimation of cell viability/cytotoxicity. Nat Protoc 3, 1125-1131 (2008). https://doi.org/10.1038/nprot.2008.75
[2] Arzneimittelforschung. 1981; 31 (8A): 1337-53; https://go.drugbank.com/drugs/DB01188
[3] EUCAST DEFINITIVE DOCUMENT E.DEF: 7.3.2. Method for the determination of broth dilution minimum inhibitory concentrations of antifungal agents for yeasts.
[4] EUCAST DEFINITIVE DOCUMENT E.DEF: 9.3.2. Method for the determination of broth dilution minimum inhibitory concentrations of antifungal agents for conidia forming moulds.
[5] Modified MIC Method for Peptides. Available online: http://cmdr.ubc.ca/bobh/method/modified-micmethod-for-cationic-antimicrobial-peptides (accessed on 5 Aug. 2021)
[6] Pilz J F, Köberle M, Kain A, Seidl P, Zink A, Biedermann T, Pilz A C. Increasing incidence of Trichophyton tonsurans in Munich-A single-centre observation. Mycoses. 2023 May; 66 (5): 441-447. doi: 10.1111/myc. 13563
[7] Macura A B, Krzyściak P, Skóra M, Gniadek A. Dermatophyte species in superficial mycoses in the Krakow district, Poland in the years 1972-2007. Mycoses. 2010 Mar. 1; 53 (2): 148-52. doi: 10.1111/j.1439-0507.2008.01684.x
[8] Michael Fehr, Zoonotic Potential of Dermatophytosis in Small Mammals, Journal of Exotic Pet Medicine, Volume 24, Issue 3, 2015, Pages 308-316, ISSN 1557-5063, https://doi.org/2015.06.015
[9] Paryuni A D, Indarjulianto S, Widyarini S. Dermatophytosis in companion animals: A review. Vet World. 2020 June; 13 (6): 1174-1181. doi: 10.14202/vetworld.2020.1174-1181
[10] Macura A B, Skóra M. 21-year retrospective study of the prevalence of Scopulariopsis brevicaulis in patients suspected of superficial mycoses. Postepy Dermatol Alergol. 2015 June; 32 (3): 189-94. doi: 10.5114/pdia.2014.40965
[11] Petanović M, Tomić Paradzik M, Kristof Z, Cvitković A, Topolovac Z. Scopulariopsis brevicaulis as the cause of dermatomycosis. Acta Dermatovenerol Croat. 2010; 18 (1): 8-13. PMID: 20361882
[12] Sellier P, Monsuez J J, Lacroix C, Feray C, Evans J, Minozzi C, Vayre F, Del Giudice P, Feuilhade M, Pinel C, Vittecoq D, Passeron J. Recurrent subcutaneous infection due to Scopulariopsis brevicaulis in a liver transplant recipient. Clin Infect Dis. 2000 May; 30 (5): 820-3. doi: 10.1086/313764
[13] Baptista P M, Vieira R, Monteiro S, Abreu A C, Gomes M, Pinto M D C. Keratitis by Scopulariopsis brevicaulis Fungus After LASIK-A Case Report. Int Med Case Rep J. 2021 Feb. 17; 14:107-110. doi: 10.2147/IMCRJ.S299454
[14] Gluck O, Segal N, Yariv F, Polacheck I, Puterman M, Greenberg D, Daniel B. Pediatric invasive sinonasal Scopulariopsis brevicaulis—a case report and 16iteraturę review. Int J Pediatr Otorhinolaryngol. 2011 July; 75 (7): 891-3. Doi: 10.1016/j.ijporl.2011.03.026
[15] Iwen P C, Schutte S D, Florescu D F, Noel-Hurst R K, Sigler L. Invasive Scopulariopsis brevicaulis infection in an immunocompromised patient and review of prior cases caused by Scopulariopsis and Microascus species. Med. Mycol. 2012 August; 50 (6): 561-9. Doi: 10.3109/13693786.2012.675629. Epub 2012 Apr. 23. PMID: 22524638]
[16] Tunç A S, Baş B, Kutsal O. Invasive Fatal Scopulariopsis brevicaulis Infection in Canaries. J Comp Pathol. 2022 August; 196:11-15. doi: 10.1016/j.jcpa.2022.06.002
[17] Sri-Jayantha L, Matthews K G, Scharf V. Scopulariopsis brevicaulis Rhinosinal Infection in a Dog. J Am Anim Hosp Assoc. 2019 January/February; 55 (1): e55102. doi: 10.5326/JAAHA-MS-6869
[18] Sridhar H, Subramanyam J R, Appaji L, Shafiulla M, Vijaykumar B R. Fusarium solani fungemia in a patient with acute lymphoblastic leukemia. Indian J Cancer. 2001 March; 38 (1): 19-21
[19] Apostolidis J, Bouzani M, Platsouka E, Belasiotou H, Stamouli M, Harhalakis N, Boutati El, Paniara O, Nikiforakis E. Resolution of fungemia due to Fusarium species in a patient with acute leukemia treated with caspofungin. Clin Infect Dis. 2003 May 15; 36 (10): 1349-50. Doi: 10.1086/374895
[20] Centers for Disease Control and Prevention (CDC). Update: Fusarium keratitis--United States, 2005-2006. MMWR Morb Mortal Wkly Rep. 2006 May 26; 55 (20): 563-4
[21] Tiribelli M, Zaja F, Fili C, Michelutti T, Prosdocimo S, Candoni A, Fanin R. Endogenous endophthalmitis following disseminated fungemia due to Fusarium solani in a patient with acute myeloid leukemia. Eur J Haematol. 2002 May; 68 (5): 314-7. doi: 10.1034/j.1600-0609.2002.01674.x
[22] Batista B G, Chaves M A, Reginatto P, Saraiva O J, Fuentefria A M. Human fusariosis: An emerging infection that is difficult to treat. Rev Soc Bras Med Trop. 2020; 53: e20200013. doi: 10.1590/0037-8682-0013-2020
[23] Nucci M, Anaissie E. Cutaneous infection by Fusarium species in healthy and immunocompromised hosts: implications for diagnosis and management. Clin Infect Dis. 2002 Oct. 15; 35 (8): 909-20. doi: 10.1086/342328
[24] Evans J, Levesque D, de Lahunta A, Jensen H E. Intracranial fusariosis: a novel cause of fungal meningoencephalitis in a dog. Vet Pathol. 2004 September; 41 (5): 510-4. doi: 10.1354/vp.41-5-510
[25] Cafarchia C, Paradies R, Figueredo L A, Iatta R, Desantis S, Di Bello A V F, Zizzo N, van Diepeningen A D. Fusarium spp. in Loggerhead Sea Turtles (Caretta caretta): From Colonization to Infection. Vet Pathol. 2020 January; 57 (1): 139-146. doi: 10.1177/0300985819880347
[26] Liu W, Feng R, Jiang H. Management of pulmonary Scedosporium apiospermum infection by thoracoscopic surgery in an immunocompetent woman. J Int Med Res. 2020 July; 48 (7): 300060520931620. doi: 10.1177/0300060520931620
[27] Chen S C, Halliday C L, Hoenigl M, Cornely O A, Meyer W. Scedosporium and Lomentospora Infections: Contemporary Microbiological Tools for the Diagnosis of Invasive Disease. J Fungi (Basel). 2021 Jan. 4; 7 (1): 23. doi: 10.3390/jof7010023
[28] Di Teodoro G, Averaimo D, Primavera M, Santoleri D, Giovannini G, Cocco A, Di Francesco G, Malatesta D, Defourny S, D'Alterio N, Curini V, Di Domenico M, Petrini A. Disseminated Scedosporium apiospermum infection in a Maremmano-Abruzzese sheepdog. BMC Vet Res. 2020 Oct. 2; 16 (1): 372. doi: 10.1186/s12917-020-02597-9
[29] Yamin D H, Husin A, Harun A. Risk Factors of Candida parapsilosis Catheter-Related Bloodstream Infection. Front Public Health. 2021 Aug. 12; 9:631865. doi: 10.3389/fpubh.2021.631865
[30] Silva S, Negri M, Henriques M, Oliveira R, Williams D W, Azeredo J. Candida glabrata, Candida parapsilosis and Candida tropicalis: biology, epidemiology, pathogenicity and antifungal resistance. FEMS Microbiol Rev. 2012 March; 36 (2): 288-305. doi: 10.1111/j.1574-6976.2011.00278.x
[31] Cordeiro R A, Sales J A, Castelo-Branco D S C M, Brilhante R S N, Ponte Y B, Dos Santos Araújo G, Mendes P B L, Pereira V S, Alencar L P, Pinheiro A Q, Sidrim J J C, Rocha M F G. Candida parapsilosis complex in veterinary practice: A historical overview, biology, virulence attributes and antifungal susceptibility traits. Vet Microbiol. 2017 December; 212:22-30. doi: 10.1016/j.vetmic.2017.07.031
[32] Maziarz E K, Perfect J R. Cryptococcosis. Infect Dis Clin North Am. 2016 March; 30 (1): 179-206. doi: 10.1016/j.idc.2015.10.006
[33] Akintilo L, Femia A. Disseminated Cryptococcosis. N Engl J Med. 2021 Oct. 28; 385 (18): 1699. doi: 10.1056/NEJMicm2109090. PMID: 34706174] [Noguchi H, Matsumoto T, Kimura U, Hiruma M, Kusuhara M, Ihn H. Cutaneous Cryptococcosis. Med Mycol J. 2019; 60 (4): 101-107. doi: 10.3314/mmj. 19.008
[34] Pennisi M G, Hartmann K, Lloret A, Ferrer L, Addie D, Belák S, Boucraut-Baralon C, Egberink H, Frymus T, Gruffydd-Jones T, Hosie M J, Lutz H, Marsilio F, Mostl K, Radford A D, Thiry E, Truyen U, Horzinek M C. Cryptococcosis in cats: ABCD guidelines on prevention and management. J Feline Med Surg. 2013 July; 15 (7): 611-8. doi: 10.1177/1098612X13489224
[35] Tini F, Covarelli L, Cowger C, Sulyok M, Benincasa P, Beccari G. Infection timing affects Fusarium poae colonization of bread wheat spikes and mycotoxin accumulation in the grain. J Sci Food Agric. 2022 November; 102 (14): 6358-6372. doi: 10.1002/jsfa.12002
[36] de Chaves M A, Reginatto P, da Costa B S, de Paschoal R I, Teixeira M L, Fuentefria A M. Fungicide Resistance in Fusarium graminearum Species Complex. Curr Microbiol. 2022 Jan. 7; 79 (2): 62. doi: 10.1007/s00284-021-02759-4
[37] Wilke A L, Bronson C R, Tomas A, Munkvold G P. Seed Transmission of Fusarium verticillioides in Maize Plants Grown Under Three Different Temperature Regimes. Plant Dis. 2007 September; 91 (9): 1109-1115. doi: 10.1094/PDIS-91-9-1109
[38] Döll S, Dänicke S. The Fusarium toxins deoxynivalenol (DON) and zearalenone (ZON) in animal feeding. Prev Vet Med. 2011 Nov. 1; 102 (2): 132-45. doi: 10.1016/j.prevetmed.2011.04.008
[39] Schoental R. Relationships of Fusarium toxins to tumours and other disorders in livestock. J Vet Pharmacol Ther. 1981 March; 4 (1): 1-6. doi: 10.1111/j.1365-2885.1981.tb00702.x
[40] Toxins derived from Fusarium graminearum, F. culmorum and F. crookwellense: zearalenone, deoxynivalenol, nivalenol and fusarenone X. IARC Monogr Eval Carcinog Risks Hum. 1993; 56:397-444. PMID: 8411625; PMCID: PMC7681618.

1. A cationic polymer with molecular weight above 13 kDa prepared based on the [2-(methacryloxy)ethyl]trimethylammonium chloride monomer having a formula: 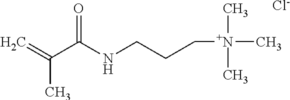
useful in the treatment or prevention of diseases caused by fungi.

2. The cationic polymer for use of claim 1, characterized in that it has mean molecular weight between 13 kDa and 892 kDa, preferably between 29 and 892 kDa.

3. The cationic polymer for use of claim 1, characterized in that the fungus is preferably of a species selected from: dermatophytes of the genus Trichophyton, in particular Trichophyton interdigitale, Trichophyton mentagrophytes, Trichophyton tonsurans or Trichophyton rubrum, Scopulariopsis brevicaulis, species of the genus Fusarium, in particular Fusarium graeminarum, Fusarium oxysporum, Fusarium proliferatum, Fusarium solani, Fusarium verticilloides, Scedosporium apiospermum, Lomentospora prolificans, Candida parapsilosis, Cryptococcus neoformans.
